(19) United States
(12) Patent Application Publication
(10) Pub. No.: US 2024/0008563 A1
(43) Pub. Date: Jan. 11, 2024

(54) LEGWEAR ITEM FOR MASSAGING AND COMPRESSING

(71) Applicant: c/o Edelweiss basics GmbH & Co. KG, Brannenburg (DE)

(72) Inventor: Georg Weber-Unger, Kufstein (AT)

(21) Appl. No.: 18/215,410

(22) Filed: Jun. 28, 2023

(30) Foreign Application Priority Data
Jul. 7, 2022 (EP) 22183621.6

(51) Int. Cl. A41D 1/06; A41F 9/00

(52) U.S. Cl. CPC A41D 1/06; A41F 9/00; A41D 2400/322

(57) ABSTRACT

The present invention relates to a legwear item. The legwear item (1) is made of three or more different cloth materials, comprising a waistband (2), a front panel (3), a rear panel (5), two side panels (4a, 4b), two knee hollow panels (6a, 6b), and two calf panels (7a, 7b), the front panel (3), the side panels (4a, 4b), and the rear panel (5) each being connected to the waistband (2), characterized in that the two side panels (4a, 4b), the rear panel (5), and the two calf panels (7a, 7b) each comprise a first cloth material for massaging and compressing leg regions of a wearer, and that the front panel (3) and the knee hollow panels (6a, 6b) each comprise a second cloth material, and that the waistband (2) comprises a third cloth material, wherein the first cloth material has a greater compression than the second cloth material.

The present invention relates to a legwear item for massaging and compressing bodily regions of a wearer.

Compression clothing is typically understood to be specialized athletic clothing worn very close to the body and acting on the body. The compression clothing exerts pressure on the musculature and veins and ensures that the blood flows better and faster. Said better and faster blood flow pumps more oxygen into the musculature. Compressing and/or massaging are applied as therapeutic measures for lymphedema, among other things, typically by means of a compression stocking.

One disadvantage of the prior art is that compression clothing brings about a uniform pressure on and thus a uniform compression of the body or parts of the body of the wearer. The wearing comfort can be adversely affected, however, because pressure is applied even to body regions where compression is perceived as uncomfortable.

A further disadvantage is that compression clothing typically leads to increased sweating of the wearer due to the tight fit on the skin. The reason is that the tight fight of the compression clothing limits the ventilation of the skin.

The object of the invention is to provide a legwear item for reducing or even eliminating the disadvantages stated above. It is therefore an object of the invention to provide a clothing item for the leg region comprising both the necessary compression and simultaneously great wearing comfort for the wearer.

A further object of the invention is to provide a legwear item also having a good massaging effect and providing support.

All of the stated objects are achieved by the legwear item according to the invention.

The invention is based on the idea that the objects stated above can be achieved by a legwear item, wherein the legwear item comprises or is made of three or more different cloth materials, particularly three or more functionally different cloth materials, particularly three functionally different cloth materials, namely a first cloth material, a second cloth material, and a third cloth material. Functionally different means that the respective cloth materials differ from each other in the compression properties, stretching properties, grammage, air-permeability, surface structure, fiber properties, and/or yarn properties thereof. The three or more cloth materials are thus different.

The invention thus provides a legwear item, preferably made of three or more different cloth materials, comprising or made of a waistband, a front panel, a rear panel, two side panels, two knee hollow panels, and two calf panels, the front panel, the side panels, and the rear panel each being connected to the waistband, characterized in that the two side panels, the rear panel, and the two calf panels each comprise a first cloth material for massaging and compressing leg regions of a wearer, and that the front panel and the knee hollow panels each comprise a second cloth material, and that the waistband comprises a third cloth material,
wherein the first cloth material has a greater compression than the second cloth material.

The legwear item according to the invention has numerous advantages. The three or more functionally different cloth materials are specifically processed for the different body regions in the legwear item according to the invention and act in said regions according to the functionality thereof. Typically, the legwear item comprises no more than 5 or no more than 4 functionally different cloth materials. The legwear item exerts compression by means of the first cloth material only at such regions of the legs of the wearer where such compression is also necessary or desired. Said regions are known as “problem zones” for women, such as the outer thighs and the buttocks region. Due to the compression at said regions of the legs of the wearer, not only massage and compression are exerted, but also the preferably raised structure of the first cloth material, for example in the form of napping, has an additional massaging effect. The massaging effect is particularly engaged when the wearer moves and thus the raised structure is displaced across the skin of the wearer to a certain degree and thus has a massaging effect. In addition, the compression also has an advantageous “shaping” or supporting effect on the outer thighs and the buttocks region of the wearer. Small indentations in the surface of the skin of the thighs and buttocks, such as are caused by cellulite, for example, are smoothed and concealed by means of the legwear item according to the invention. Thus in the worn state of the legwear according to the invention, there is not only a massaging and compression effect, but also an esthetic effect for the wearer at the same time. For the above reasons, the first cloth material can therefore also be referred to as a compression material.

The first cloth material or the compression material is predominantly used for covering the large muscle groups of the legs of the wearer in order to exert corresponding pressure or compression on said muscle groups, and to support said muscle groups at the same time. Said large muscle groups are present at the outer thighs, the buttocks region, and the calves of the wearer.

At the other regions of the legs, a lower compression in comparison with the first cloth material is exerted by means of the second cloth material of the legwear item, and a lower compression in comparison with the second cloth material or no compression at all is exerted by means of the third cloth material. Said regions are the knee hollow panels or the front panel for the second cloth material. Compression or pressure is perceived to be unpleasant at said regions and would therefore reduce wearing comfort. In this respect, the second cloth material can also be referred to as comfort material. The second cloth material preferably has greater breathability than the first cloth material.

The third cloth material is used for the waistband of the legwear item according to the invention. The third cloth material preferably has a velvety surface.

The third cloth material differs from the second cloth material in the elasticity thereof. In particular, the third cloth material has a greater elasticity and/or a softer or more velvety surface than the second cloth material. The third cloth material thus has the greatest comfort of all three or more cloth materials used for the legwear item. In particular, the third cloth material has greater comfort relative to both the second cloth material and the first cloth material.

The legwear item according to the invention comprises a plurality of parts, namely a waistband, a front panel, a rear panel, two side panels, two knee hollow panels, and two calf panels. The legwear item according to the invention is preferably made of a waistband, a front panel, a rear panel, two side panels, two knee hollow panels, and two calf panels.

The following definitions are used in the sense of the present application. The directional and orientational terms “proximal” and “distal” indicate, respectively, “situated toward the center of the body” and “situated away from the center of the body.” When a part of the legwear item according to the invention has a proximal end and a distal end, for example, then the proximal end of the part is closer to or in the direction of the torso of the wearer in the worn state of the legwear item, while the distal end of the part is closer to or in the direction of the foot of the wearer.

The waistband of the legwear item contacts the lower abdomen, the waist, and the lower back of the wearer in the worn state. The third cloth material, that is, the cloth material having the greatest comfort, is used for the waistband. The waistband comprises a top waistband edge and a bottom waistband edge. The top waistband edge is thereby disposed proximally, and the bottom waistband edge is disposed distally. The width of the waistband, that is, the spacing between the top waistband edge and the bottom waistband edge, is preferably between 0.5 cm and 15 cm, more preferably between 2 cm and 12 cm. The waistband of the legwear item is preferably implemented as a single piece, alternatively as two pieces. If the waistband is implemented as two pieces, then the waistband preferably comprises a front waistband part and a rear waistband part, wherein the front waistband part is connected to the rear waistband part on each side in the region of the hips and/or waist of the wearer in the worn state of the legwear item.

The front region of the waistband or rear region of the waistband means such region of the waistband contacting the belly side or the back side of the wearer in the worn state of the legwear item. A side region of the waistband thus means such region of the waistband contacting the side of the wearer in the worn state of the trousers.

A single piece in the sense of the present application means that the corresponding part is made of a single, continuous piece of a cloth material. Two pieces in the sense of the present application means that the corresponding part is made of two pieces of the same cloth material connected to each other. The connecting can thereby take place by means of sewing, needling, gluing, or other techniques known in the textiles field for permanently connecting the two pieces to each other.

The third cloth material preferably has a velvety surface, that is, the third cloth material comprises or is made of a woven fabric having a pile of protruding threads or fibers typically forming a 1 to 3 mm high tufted surface. Velvet is known in the prior art. The third cloth material is used preferably reversed as the waistband. The third cloth material preferably comprises stretch material, preferably multiway stretch material, or moleskin. A multiway stretch material can be stretched slightly in all directions. Moleskin is a cotton fabric and is also known by the name English leather.

The legwear item further comprises two side panels, namely a left side panel and a right side panel, wherein both the left and the right side panel each comprise a proximal end and a distal end. The two side panels are connected to the waistband at the proximal end of each, preferably to the bottom waistband edge of the waistband. In the worn state of the legwear item, the left side panel contacts the left leg of the wearer, and the right side panel correspondingly contacts the right leg of the wearer, and thus cover the muscle groups at the outer thighs of the wearer.

The two side panels preferably extend, starting from a side region of the waistband in each case, across the outer thigh region, to an outer side knee region of the wearer in the worn state of the legwear item, more preferably starting from a side region of the bottom waistband edge, across the outer thigh region, to at least one side knee region of the wearer. The two side panels are thereby each connected at the corresponding distal ends thereof to the corresponding front panels, more preferably to the corresponding front panels and to the corresponding knee hollow panels at the same time.

The two side panels preferably each have a band-shaped design and/or the two side panels are each implemented as a single piece.

The legwear item further comprises a front panel, wherein the front panel comprises a proximal end and a distal end. The comfort material, that is, the second cloth material, is used for the front panel.

The front panel preferably extends, starting from a front region of the waistband, preferably from a front region of the bottom waistband edge, across the crotch region, the two front thigh regions and inner thigh region, to at least a front knee region of the wearer in each case in the worn state of the legwear item, more preferably each at least to an ankle region of the wearer. If the waistband is implemented as two pieces, then the front panel preferably extends, starting from a front waistband part, more preferably from a bottom edge of the front waistband part, across the two front thigh regions, in each case at least to a front knee region of the wearer in the worn state, more preferably in each case at least to an ankle region of the wearer.

The front panel can be implemented as a single piece or as two pieces. If the front panel is implemented as two pieces, then the front panel comprises a left front panel and a right front panel implemented symmetrically to each other. The left and the right front panel are connected to each other along a medial line running from a crotch region of the legwear item to the bottom waistband edge, preferably to the bottom waistband edge of the front waistband part. The connecting can take place by means of sewing, needling, gluing, or other techniques known in the textiles field for permanently connecting the two pieces to each other.

The legwear item further comprises a rear panel. The rear panel is preferably implemented as a single piece, but can also be implemented as two pieces. The compression material, that is, the first cloth material, is used for the rear panel for the purpose of compressing the buttocks muscles of the wearer.

The rear panel preferably extends, starting from a rear region of the waistband, preferably from a rear region of the bottom waistband edge, across a buttocks region and the two rear thigh regions of the wearer, to the corresponding knee hollow panels of the legwear item in the worn state of the legwear item. The rear panel is preferably connected to the two knee hollow panels. In other words, the corresponding distal ends of the rear panel extend to the proximal ends of the corresponding knee hollow panels and are connected to the same. The connecting can take place by means of sewing, needling, gluing, or other techniques known in the textiles field for permanently connecting the two pieces to each other.

The rear panel can be implemented as a single piece or as two pieces. If the rear panel is implemented as two pieces, then the rear panel comprises a left rear panel and a right rear panel implemented symmetrically to each other. The left and the right rear panel are connected to each other along a medial line running from a crotch region of the legwear item to the bottom waistband edge, preferably to the bottom waistband edge of the rear waistband part. The connecting can take place by means of sewing, needling, gluing, or other techniques known in the textiles field for permanently connecting the two pieces to each other.

The legwear item further comprises two knee hollow panels, namely a left knee hollow panel and a right knee hollow panel, wherein both the left and the right knee hollow panel each comprise a proximal end and a distal end. The left and the right knee hollow panel are preferably implemented symmetrically to each other. The comfort material, that is, the second cloth material, is used for the knee hollow panels.

In the worn state of the legwear item, the left knee hollow panel makes contact with the left leg of the wearer in the region of the hollow of the knee, and the right knee hollow panel correspondingly makes contact with the right leg of the wearer in the region of the hollow of the knee. The two knee hollow panels preferably each also extend across the lower, rear thigh regions of the wearer and/or the two knee hollow panels also extend across the upper calf regions of the wearer.

The proximal ends of the knee hollow panels are preferably each connected to the distal end of the rear panel, and each distal end of the knee hollow panels is connected to the corresponding calf panel. The connecting can take place by means of sewing, needling, gluing, or other techniques known in the textiles field for permanently connecting the two pieces to each other.

The legwear item further comprises two calf panels, namely a left calf panel and a right calf panel, wherein both the left and the right calf panel each comprise a proximal end and a distal end. The compression material, that is, the first cloth material, is used for the calf panel for the purpose of compressing the calf muscles of the wearer.

In the worn state of the legwear item, the left calf panel makes contact with the left leg of the wearer in the calf region, and the right calf panel correspondingly makes contact with the right leg of the wearer in the calf region. The corresponding calf muscles of wearer are thus covered and compressed by means of the calf panels.

The proximal ends of the calf panels are preferably each connected to the distal end of the corresponding knee hollow panel. The connecting can take place by means of sewing, needling, gluing, or other techniques known in the textiles field for permanently connecting the two pieces to each other.

The first cloth material preferably comprises a raised structure and the raised structure of the first cloth material directly contacts the skin of the wearer of the legwear item in the worn state, and/or the second cloth material comprises a smooth structure, and the smooth structure of the second cloth material directly contacts the skin of the wearer of the legwear item in the worn state. Directly contacts means that the corresponding cloth material makes direct contact with the skin of the wearer, that is, there is no cloth layer or the like between the skin of the wearer and the corresponding cloth material.

The first cloth material, preferably having a raised structure, preferably differs from the second cloth material in the compression, stretching, weight, fiber properties, and/or yarn properties thereof. In particular, the first cloth material has a greater compression and/or greater stretching than the second cloth material.

The first cloth material, that is, the compression material, preferably comprises polyamide, viscose, or polyester, more preferably a combination of polyamide having 1-30% elastane or a combination of polyester having 1-30% elastane. The elastane proportion can be adjusted within the indicated range according to need with respect to elasticity and compression properties of the first cloth material.

The second cloth material, that is, the comfort material, preferably comprises cotton or stretch material, more preferably the second cloth material comprises piqué or a multiway stretch material. Cotton, particularly as piqué, is breathable and is therefore particularly suitable as a comfort material. A multiway stretch material can be stretched slightly in all directions.

The raised structure of the first cloth material is preferably implemented in the form of napping. The napping is thereby preferably disposed on the cloth material in a regular or an irregular pattern. The napping can have any arbitrary shape, for example circular napping or three-side, four-sided, or multi-sided napping. The napping can be weft knitted or warp knitted.

Each calf panel preferably extends from the corresponding knee hollow panel to a trouser leg hem in the worn state.

The front panel and the corresponding calf panel each preferably form a common trouser leg hem, or the two distal ends of the front panel and the corresponding distal ends of the two calf panels more preferably form a common trouser leg hem of the legwear item.

The front panel, the corresponding knee hollow panel, and the corresponding calf panel each preferably form a common trouser leg hem; the two distal ends of the front panel and the corresponding distal ends of the two knee hollow panels and the corresponding distal ends of the two calf panels more preferably form a common trouser leg hem of the legwear item.

The legwear item is preferably a pair of leggings or trousers. The trousers are preferably running trousers or sports trousers. The trousers, particularly running trousers or sports trousers, can be short, half-length, or long trousers, particularly running trousers or sports trousers.

The trouser leg hem is preferably present in the region of the hollow of the knee of the wearer. In this case, the trousers are implemented as half-length trousers.

The trouser leg hem is preferably present in the region of the calf of the wearer. In this case, the trousers are implemented as half-length trousers.

The trouser leg hem is preferably present in the region of the ankle of the wearer. In this case, the trousers are implemented as long trousers.

The invention is described in greater detail below using the attached figures. Shown are:

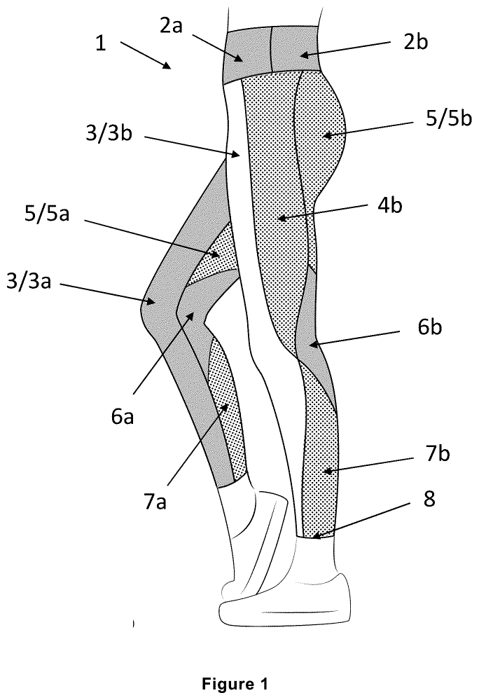

FIG. 1 a side view of the legwear item according to one embodiment of the invention,

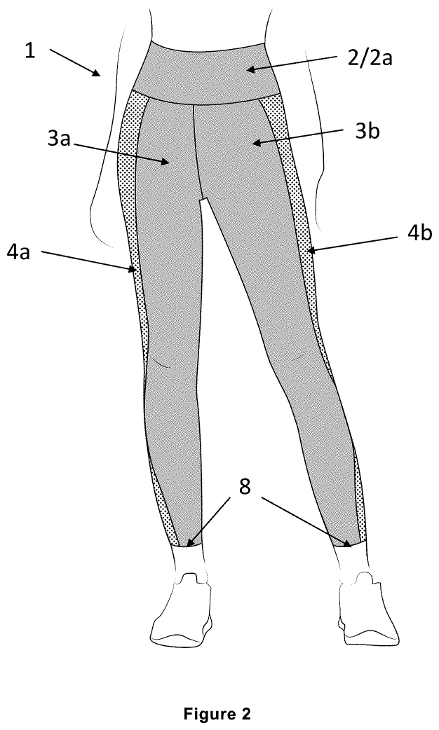

FIG. 2 a front view of the legwear item according to the embodiment of FIG. 1,

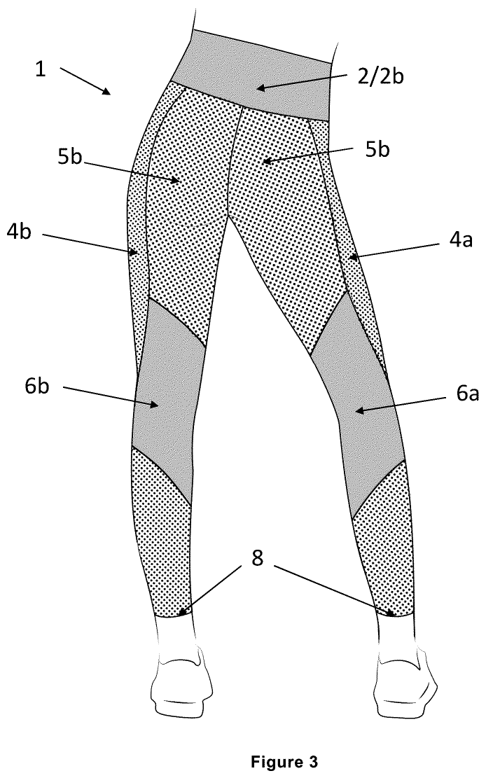

FIG. 3 a rear view of the legwear item according to the embodiment of FIG. 1.

### REFERENCE NUMERALS

1 Legwear item
2 Waistband
2a Front waistband part
2b Rear waistband part
3 Front panel
3a Right front panel
3b Left front panel
4a Right side panel
4b Left side panel
5 Rear panel
5a Right rear panel
5b Left rear panel
6a Right knee hollow panel
6b Left knee hollow panel
7a Right calf panel
7b Left calf panel
8 Trouser leg hem

The legwear item (1) of the embodiment example of the invention shown in FIGS. 1 through 3 is implemented in the form of skintight leggings. Said item comprises a two-piece waistband (2) having a front waistband part (2a) and a rear waistband part (2b). As can be seen in FIG. 1, the front waistband part (2a) is sewn at the side to the rear waistband part (2b) in the region of the hip of the wearer. The waistband (2) is band-shaped in design and contacts the waist and hip region of the wearer. The waistband width, that is, the spacing between a top and a bottom waistband edge of the waistband (2), is approximately 10 cm.

The legwear item (1) further comprises a front panel (3) implemented here as a two-piece front panel, namely a right front panel (3a) and a left front panel (3b) implemented symmetrically to each other. The front panel (3) extends, starting from the bottom waistband edge of the waistband (2), across the front thigh regions and the front knee region, to an ankle region of the wearer.

The legwear item further comprises two side panels, namely a right side panel (4a) and a left side panel (4b), also implemented symmetrically to each other. The two side panels each extend from the bottom waistband edge of the waistband (2), across the sides of the thighs, to a side knee region of the wearer.

The legwear item (1) further comprises a rear panel (5). The rear panel (5) is implemented in two pieces and comprises a left rear panel (5b) and a right rear panel (5a). The two rear panels each extend from the bottom waistband edge of the waistband (2), across the buttocks of the wearer, to near the hollow of the knee of the wearer. The distal ends of the rear panels are sewn there to the proximal ends of the corresponding knee hollow panels (6a, 6b). The distal ends of the knee hollow panels (6a, 6b) are sewn to the proximal ends of the corresponding calf panels (7a, 7b).

Together with the two calf panels (7a, 7b), the corresponding front panels (3a, 3b) form the two trouser leg hems (8).

The two-piece front panel (3a, 3b) and the two knee hollow panels (6a, 6b) are made of piqué, that is, the second cloth material. The stitched surface of the piqué is disposed toward the outside, while the smooth surface of the piqué faces toward the skin of the wearer.

The waistband (2) is made of a third cloth material, in this case a multiway stretch material having a velvety surface.

In contrast, the two side panels (4a, 4b), the two rear panels (5a, 5b), and the two calf panels (7a, 7b) are made of the first cloth material, that is, a compression material. In the present case, the first cloth material is a mixture of polyamide and elastane.

1. A legwear item made of three or more different cloth materials and comprising a waistband, a front panel, a rear panel, two side panels, two knee hollow panels, and two calf panels, the front panel, the side panels (4a, 4b), and the rear panel each being connected to the waistband, characterized in that the two side panels, the rear panel, and the two calf panels each comprise a first cloth material for massaging and compressing leg regions of a wearer, and that
the front panel and the knee hollow panels each comprise a second cloth material, and that
the waistband comprises a third cloth material,
wherein the first cloth material has a greater compression than the second cloth material.

2. The legwear item according to claim 1, characterized in that the first cloth material comprises a raised structure and the raised structure of the first cloth material directly contacts the skin of the wearer of the legwear item in the worn state, and/or that the second cloth material comprises a smooth structure, and the smooth structure of the second cloth material directly contacts the skin of the wearer of the legwear item in the worn state.

3. The legwear item according to claim 1, characterized in that the first cloth material comprises polyamide, viscose, or polyester, and/or that the second cloth material comprises cotton or stretch material, preferably multiway stretch material.

4. The legwear item according to claim 1, characterized in that the second cloth material has greater breathability than the first cloth material.

5. The legwear item according to claim 1, characterized in that the third cloth material comprises a velvety surface.

6. The legwear item according to claim 1, characterized in that the first cloth material, the second cloth material, and the third cloth material differ from each other in the compression properties, stretching properties, grammage, air-permeability, surface structure, fiber properties, and/or yarn properties thereof.

7. The legwear item according to claim 2, characterized in that the raised structure of the first cloth material is implemented in the form of napping.

8. The legwear item according to claim 1, characterized in that the front panel extends in the worn state from a front region of the waistband, across the crotch region, the two front thigh regions and inner thigh regions, to in each case at least one front knee region of the wearer.

9. The legwear item according to claim 1, characterized in that the rear panel extends in the worn state from a rear region of the waistband, across a buttocks region and the two rear thigh regions of the wearer, to the corresponding knee hollow panels.

10. The legwear item according to claim 1, characterized in that the side panels extend in the worn state from a side region of the waistband, across the outer thigh region, to an outer side knee region of the wearer.

11. The legwear item according to claim 1, characterized in that the two side panels each have a band-shaped design.

12. The legwear item according to claim 1, characterized in that the two side panels are each implemented as a single piece.

13. The legwear item according to claim 1, characterized in that the front panel is implemented in two pieces and/or that the rear panel is implemented in two pieces.

14. The legwear item according to claim 1, characterized in that the front panel and the corresponding calf panel each form a common trouser leg hem, or that the front panel, the corresponding knee hollow panel, and the corresponding calf panel each form a common trouser leg hem.

15. The legwear item according to claim 14, characterized in that the trouser leg hem is present in the region of the calf of the wearer, or that the trouser leg hem is present in the region of the ankle of the wearer.
